(19) United States
(12) Patent Application Publication
(10) Pub. No.: US 2026/0294682 A1
(43) Pub. Date: Oct. 1, 2026

(54) DYNAMICALLY RETRACTABLE STIFFENERS FOR SURGICAL TOOLS

(71) Applicant: Alcon Inc., Fribourg (CH)

(72) Inventors: Bill Chen, Irvine, CA (US); Robert Y. Diehl, Dana Point, CA (US)

(21) Appl. No.: 19/542,959

(22) Filed: Feb. 18, 2026

Related U.S. Application Data
(60) Provisional application No. 63/777,849, filed on Mar. 26, 2025.

(51) Int. Cl. A61F 9/007

(52) U.S. Cl. CPC A61F 9/00736

(57) ABSTRACT

A surgical tool comprising a body, a needle extending from a distal end of the body to access a surgical area, and a stiffening sleeve extending from the distal end and surrounding at least a portion of the needle for stabilizing the needle. The surgical tool further comprises a first magnet coupled to a proximal end of the stiffening sleeve and a second magnet within the body. The first magnet and the second magnet are arranged such that a repulsive force is formed between the first magnet and the second magnet. When external proximal forces on the stiffening sleeve are less than the repulsive force, the first magnet and the stiffening sleeve are in a fully extended position, and when external proximal forces on the stiffening sleeve are greater than the repulsive force, the first magnet and the stiffening sleeve are retracted proximally towards the body.

### INTRODUCTION

Continuous efforts to minimize the invasiveness of surgical procedures, such as ophthalmic surgical procedures, have led to the development of small-gauge surgical instrumentation for microincision techniques. Small gauge vitrectomy, also known as minimally invasive vitreous surgery (MIVS), is a classic example of one such type of surgical procedure utilizing small-gauge instrumentation. Examples of common ocular conditions that may be treated by minimally invasive vitreous surgery include retinal detachment, macular holes, premacular fibrosis, and vitreous hemorrhages. The benefits associated with modern MIVS as compared to more invasive vitrectomies include access to greater pathology, greater fluidic stability, increased patient comfort, less conjunctival scarring, less postoperative inflammation, and earlier visual recovery, among others. Accordingly, indications for MIVS and other microincision techniques have expanded in recent years.

Despite the aforementioned benefits of microincision techniques and their widespread acceptance, there remain numerous challenges with the utilization of small-gauge surgical instruments, particularly in the field of ophthalmology. One commonly noted concern among surgeons is instrument rigidity. The smaller diameter of these microincision instruments, such as vitrectomy probes and illuminators, causes decreased stiffness thereof, making it difficult for surgeons to control the instruments during certain ocular surgical procedures. With small gauge ophthalmic surgical instruments, for example, the instrument tips can move in unintended directions at the extreme limits of the eye, thus making delicate procedures such as the peeling of membranes from the retinal surface extremely difficult. Additionally, the decreased stiffness of small-gauge surgical instruments makes such instruments susceptible to unintended damage when being handled outside of the eye.

Accordingly, what is needed in the art are improved methods and apparatus for stiffening small-gauge surgical instruments.

### SUMMARY

The present disclosure generally relates to methods and apparatuses for ophthalmic surgical procedures.

Certain embodiments of the present disclosure provide a surgical tool. The surgical tool comprises a surgical tool body comprising a proximal end and a distal end, a needle extending from the distal end of the surgical tool body configured to access a surgical area within a body of a patient, and a stiffening sleeve extending from the distal end of the surgical tool body and surrounding at least a portion of the needle for stabilizing the needle during a surgical procedure. The surgical tool further comprises a first magnet coupled to a proximal end of the stiffening sleeve and a second magnet disposed within the surgical tool body. The first magnet and the second magnet are arranged such that a repulsive force is formed between the first magnet and the second magnet. When external proximal forces on the stiffening sleeve are less than the repulsive force, the first magnet and the stiffening sleeve are in a fully extended position, and when external proximal forces on the stiffening sleeve are greater than the repulsive force, the first magnet and the stiffening sleeve are retracted proximally towards the surgical tool body.

Certain embodiments of the present disclosure provide a surgical tool. The surgical tool comprises a surgical tool body comprising a proximal end and a distal end, a needle extending from the distal end of the surgical tool body configured to access a surgical area within a body of a patient, and a telescopic stiffening sleeve extending from the distal end of the surgical tool body and surrounding at least a portion of the needle for stabilizing the needle during a surgical procedure. The telescopic stiffening sleeve comprises at least a first segment and a second segment. The surgical tool further comprises a biasing mechanism disposed within the surgical tool body and extending with the telescopic stiffening sleeve, wherein the biasing mechanism is configured to provide a distal biasing force against the telescopic stiffening sleeve. When external proximal forces on the telescopic stiffening sleeve are less than the distal biasing force, the telescopic stiffening sleeve is fully extended, and when external proximal forces on the telescopic stiffening sleeve are greater than the distal biasing force, the telescopic stiffening sleeve is retracted proximally towards the surgical tool body. When the telescopic stiffening sleeve is retracted proximally, the first segment is configured to at least partially nest within the second segment.

The following description and the related drawings set forth herein detail certain illustrative features of one or more embodiments, including those described above.

### BRIEF DESCRIPTION OF THE DRAWINGS

The drawings described herein are for illustrative purposes only, are schematic in nature, and are intended to be exemplary rather than to limit the scope of the disclosure.

FIG. 1 illustrates a perspective view of a surgical tool comprising a needle aided by a dynamically retractable stiffening sleeve, according to embodiments described herein.

FIGS. 2A-2B illustrate cross-sectional side views of a portion of a surgical assembly including a needle and a stiffening sleeve positioned outside of a cannula assembly and entering a cannula assembly, respectively, according to embodiments described herein.

FIG. 3A illustrates a cross-sectional perspective view of a portion of an example surgical tool including a magnet-biased stiffening sleeve, according to embodiments described herein.

FIG. 3B illustrates a cross-sectional side view of a portion of an example surgical tool including a magnet-biased stiffening sleeve in an extended state, according to embodiments described herein.

FIG. 3C illustrates a cross-sectional side view of a portion of an example surgical tool including a magnet-biased stiffening sleeve in a retracted state, according to embodiments described herein.

FIG. 4A illustrates an isometric view of a portion of an example surgical tool having a telescopic stiffening sleeve in an extended state, according to embodiments described herein.

FIG. 4B illustrates an isometric view of a portion of an example surgical tool having a telescopic stiffening sleeve in a retracted state, according to embodiments described herein.

FIG. 5A illustrates a cross-sectional side view of a portion of an example surgical tool having a telescopic stiffening sleeve in an extended state, according to embodiments described herein.

FIG. 5B illustrates a magnified view of a portion of the example surgical tool of FIG. 5A, according to embodiments described herein.

FIG. 5C illustrates a cross-sectional side view of a portion of an example surgical tool having a telescopic stiffening sleeve in a retracted state, according to embodiments described herein.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

It will be readily understood that the components of the embodiments as generally described herein and illustrated in the appended Figures can be arranged and designed in a wide variety of different configurations. Thus, the following more detailed description of various embodiments, as represented in the Figures, is not intended to limit the scope of the present disclosure but is merely representative of various embodiments. While the various aspects of the embodiments are presented in the Figures, the Figures are not necessarily drawn to scale unless specifically indicated.

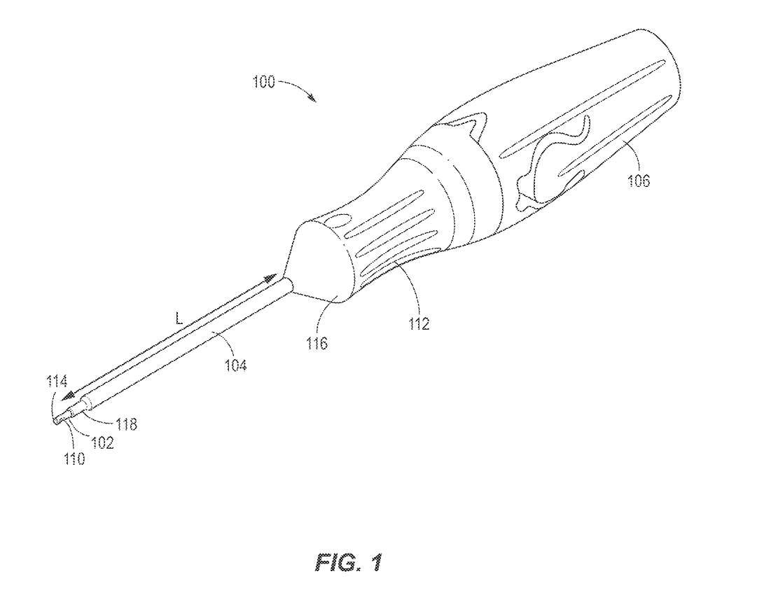

Embodiments herein are described with reference to certain types of ophthalmic surgical procedures. In particular, a vitrectomy tool in which vitreous humor is removed to address, e.g., retinal detachment, diabetic retinopathy, macular holes, vitreous floaters, vitreous hemorrhage, etc., is illustrated in FIG. 1. However, tools and techniques detailed herein may be employed in a variety of other devices, manners, and/or procedures as well. For example, a needle or other implement reaching into an eye for an ophthalmic surgical procedure may include a forceps, scissors, spatula, knife, etc. Further, while a vitrectomy procedure is largely described herein, embodiments of a vitrectomy tool as detailed herein may be utilized in conjunction with other ophthalmic tools to address a variety of eye conditions. Additionally, while vitrectomy and other ophthalmic surgeries often benefit from the use of fairly thin needles, other types of surgeries (e.g., other than eye surgeries) may benefit from the unique architecture and techniques detailed herein. Indeed, so long as a supportive stiffening sleeve is employed that is dynamically retractable during surgery, appreciable benefit may be realized.

Note that, as described herein, a “distal” end, side, or portion of a component refers to the end, side, or the portion that is closer to the patient’s body during the use thereof. On the other hand, a “proximal” end, side, or portion of the component refers to the end, side, or portion that is distanced further away from the patient’s body.

FIG. 1 illustrates a perspective view of a surgical tool 100 comprising a needle 102 aided by a stiffening sleeve 104, according to embodiments described herein. As shown, surgical tool 100 is a vitrectomy tool (i.e., a vitrectomy probe) for use in vitrectomy procedures. However, other surgical tools, such as an illumination probe, forceps, scissors, etc., can incorporate the stiffening sleeves and stiffening sleeve actuation mechanisms described herein.

The surgical tool 100 includes a body 106 having a gripping element 112 configured to be held by a user (e.g., a surgeon) during handling and/or performance of the vitrectomy procedure. The needle 102 extends from a distal end 116 of the body 106. In the exemplary embodiments, the needle 102 can support a cutter therein, which cuts vitreous and/or other tissues aspirated through a port 110 at a distal end 114 of the needle 102. A proximal end of the needle 102 is disposed within the body 106.

The needle 102 is generally small in outer diameter to facilitate performance of minimally invasive vitreous surgery (MIVS) (e.g., 20 gauge, 23 gauge, 25 gauge, 27 gauge, or smaller). The needle 102 can be formed of a surgical grade metallic material, such as stainless steel, aluminum, titanium, nitinol, etc. However, even when made of a metallic material, the small diameter of the needle 102 may cause the needle 102 to be flexible and susceptible to extreme bending when being handled by the surgeon or other operating staff prior to and during use in a surgical procedure. The flexibility of the needle 102 may make handling of the surgical tool 100 more difficult, such as by creating difficulty when aligning and inserting the needle 102 into a cannula, or by causing unintended movement of the needle 102 when disposed through the cannula and in a patient’s eye, for example.

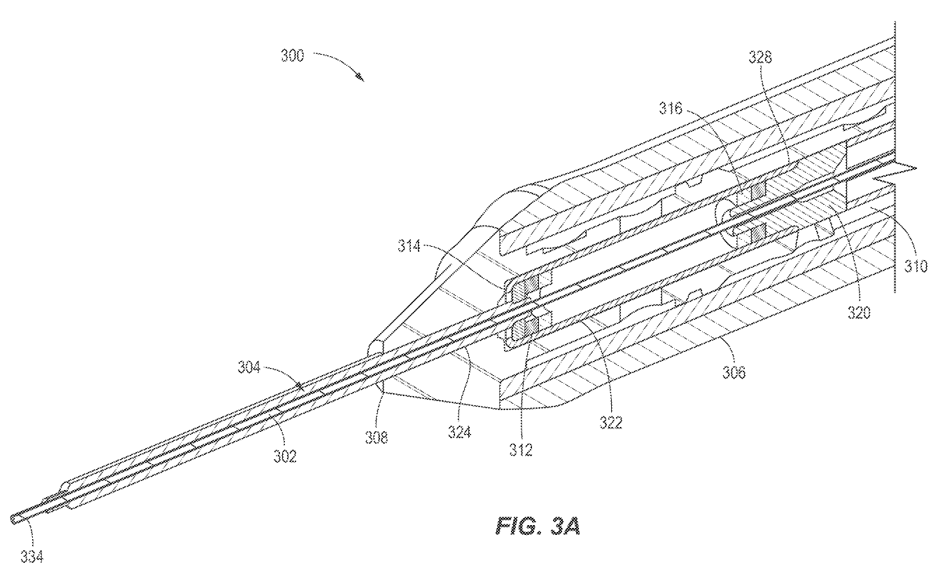

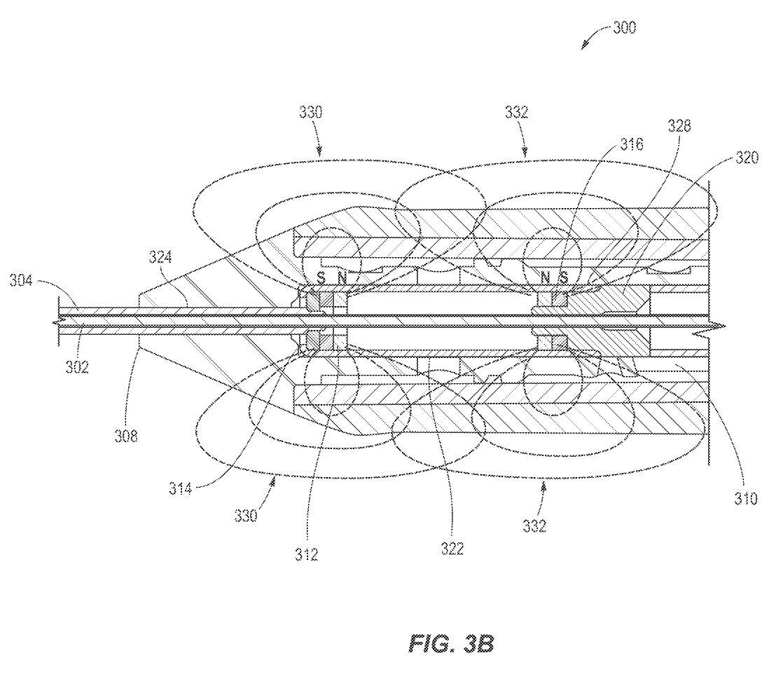

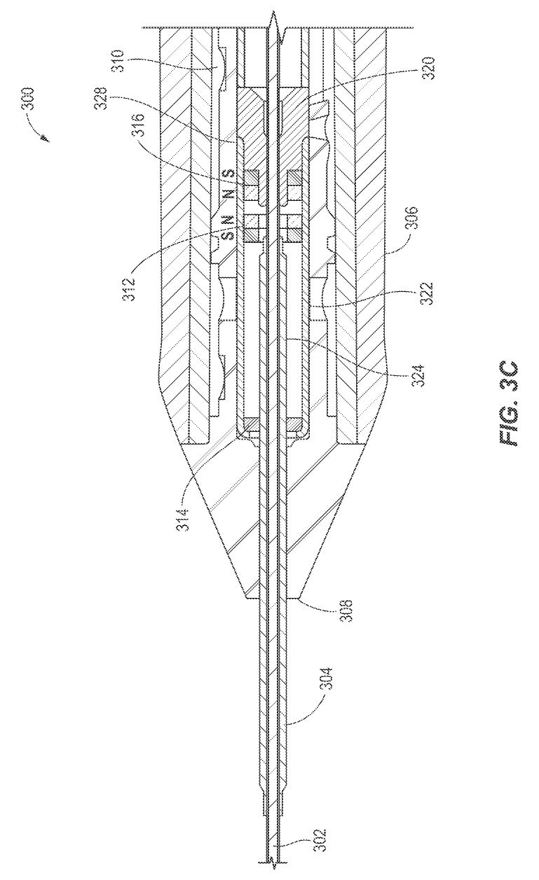

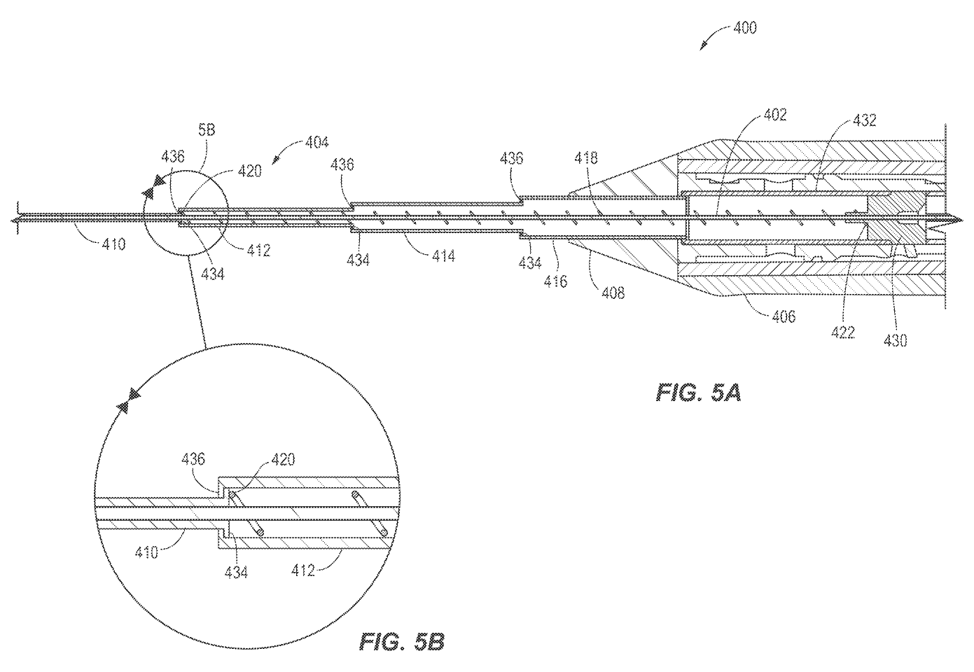

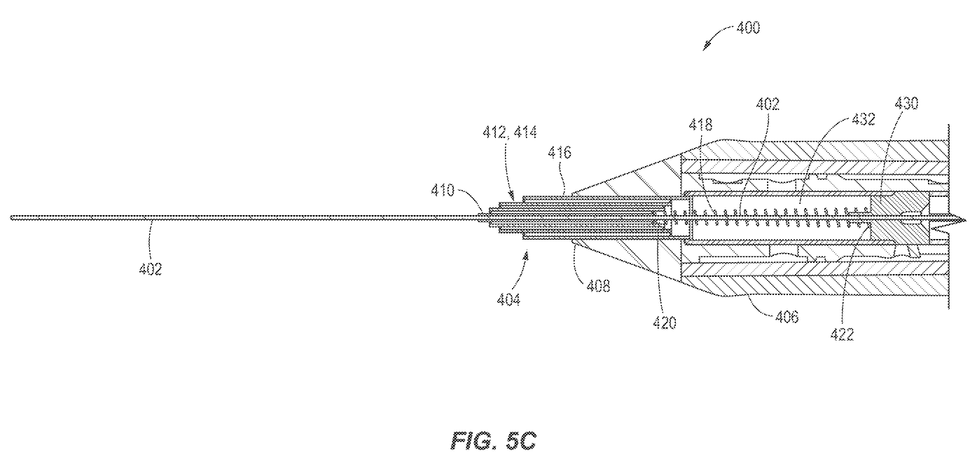

Therefore, in order to protect the needle 102 and facilitate a desired level of control over the needle 102 during handling of the surgical tool 100, a stiffening sleeve 104 is slidably disposed around the needle 102. The stiffening sleeve 104 is generally cylindrical in shape. The stiffening sleeve 104 is configured to extend and retract along the needle 102 such that a length L of the needle 102, external to the body 106 and over which the stiffening sleeve 104 extends, can be adjusted. The stiffening sleeve 104 can be dynamically retracted, without direct manipulation from the user, between a fully extended state and a fully retracted state in response to external and proximal force(s) against the stiffening sleeve 104. In a fully extended state, a distal end 118 of the stiffening sleeve 104 is disposed adjacent to the distal end 114 of the needle 102. In a fully retracted state, the distal end of the stiffening sleeve 104 is disposed adjacent the distal end 116 of the body 106. The surgical tool 100 further includes a biasing mechanism (examples shown in FIGS. 3A-3C and 5A-5C) coupled to the stiffening sleeve 104 that provides proximal biasing force(s) against the stiffening sleeve 104 to extend the stiffening sleeve 104, which can be overcome by the external force(s) to retract the stiffening sleeve 104.

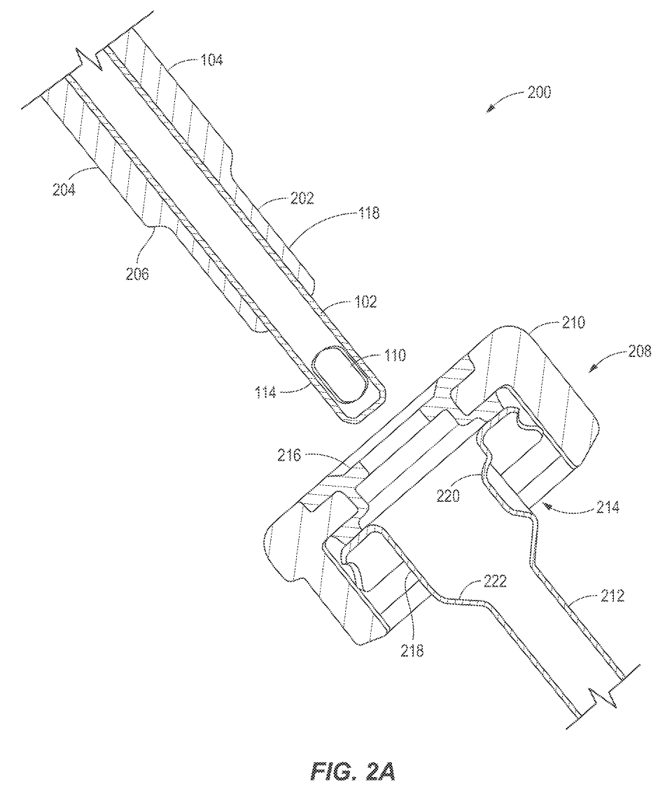

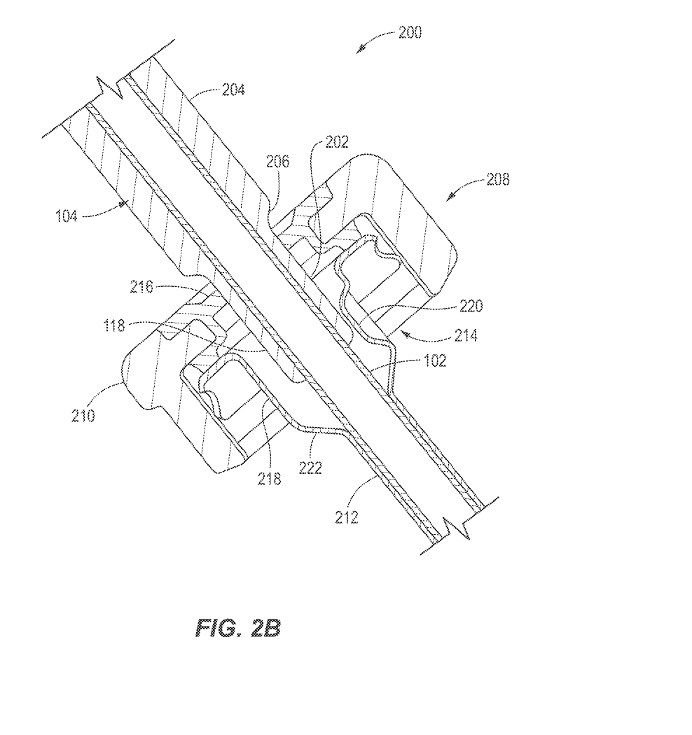

In certain embodiments, the amount, or extent, of retraction of the stiffening sleeve 104 is based on the amount, or magnitude, of external force(s) and/or biasing force(s) acting on the stiffening sleeve 104, as further described in reference to FIGS. 2A-2B. As described herein, the stiffening sleeve 104, and specifically the ability of the stiffening sleeve 104 to dynamically retract in response to the external force(s), enables: (1) the needle 102 to be protected and its rigidity maximized along at least a portion of the needle 102 prior to being inserted into a patient’s eye (e.g., when at least a portion of the needle 102 is sheathed by the stiffening sleeve 104), (2) the stiffening sleeve 104 to efficiently and readily retract when inserting the needle 102 into the patient’s eye without additional input or manipulation by the user, and (3) focused protection and improved stiffening at a proximal portion of the needle 102 external to the patient’s eye after the needle 102 is inserted into the eye.

In certain embodiments, the stiffening sleeve 104 can be formed of a metallic material such as surgical grade stainless steel, aluminum, or the like. In certain embodiments, the stiffening sleeve 104 can be formed of a thermoplastic polymeric material, such as high-density polyethylene (HDPE) or the like.

While the stiffening sleeve 104 is illustrated as a unitary structure, the stiffening sleeve 104 can include separate segments fixedly or dynamically coupled to one another to sheathe the needle 102. In such an example, each segment can have a length equivalent to only a portion of the length of the needle 102.

FIGS. 2A-2B illustrate cross-sectional side views of a portion of a surgical assembly 200 including the surgical tool 100 and a cannula assembly 208, according to embodiments described herein. FIG. 2A depicts the needle 102 of the surgical tool 100 disposed outside the cannula assembly 208 (e.g., prior to be inserted into an eye of a patient), while FIG. 2B depicts the needle 102 inserted into the cannula assembly 208 (e.g., for insertion into the eye of the patient).

As shown in FIG. 2A, the stiffening sleeve 104 is disposed around the needle 102 and is configured to slide along the needle 102 as a result of the application of external proximal forces on the stiffening sleeve 104, and/or internal and distal biasing forces provided by a biasing mechanism configured to counter the external forces. In certain embodiments, the stiffening sleeve 104 includes at least a first portion 202 of reduced thickness at a most distal position at the distal end 118, and a second portion 204 having a thickness greater than the first portion 202 and positioned proximal to the first portion 202. The reduced thickness of the stiffening sleeve 104 at the first portion 202 results in the first portion 202 having a smaller outer diameter than an outer diameter of the second portion 204. In such embodiments, the smaller outer diameter of the first portion 202 facilitates easier insertion of the surgical tool 100 into the cannula assembly 208. In certain embodiments, a transition 206 extends between first portion 202 and the second portion 204. The transition 206 can include an outer surface that is sloped, tapered, or otherwise variable in profile as compared to outer surfaces of the first portion 202 and/or second portion 204.

The cannula assembly 208 includes a hub 210 coupled to a head 218 of a cannula 212. The cannula assembly 208 is configured to be inserted into a patient’s eye during an ophthalmic procedure to facilitate access to a surgical site within the eye. For example, at the start of the surgical procedure, a trocar blade disposed through the cannula assembly 208 is used to create a micro-incision within the patient’s eye. Once the incision is made, the cannula 212 is inserted into the incision in the patient’s eye and the trocar blade is removed from the cannula assembly 208. When the cannula 212 is inserted into the incision in the patient’s eye, a distal portion 214 of the hub 210 rests on an external surface of the patient’s eye. The surgical tool 100 can then be inserted through the hub 210 and through the cannula 212 to gain access to the surgical site within the patient’s eye.

When the user inserts the surgical tool 100 into the cannula assembly 208, as shown in FIG. 2B, the distal end 114 of the needle 102 and at least the first portion 202 of the stiffening sleeve 104 are translated through the hub 210 and into the head 218 of the cannula 212. As the user further translates the surgical tool 100 distally into the eye through the cannula assembly 208, as shown in FIG. 2B, the transition 206 of the stiffening sleeve 104 engages with a diaphragm 216 attached to the hub 210. The transition 206 can act as a “stop” for the stiffening sleeve 104, preventing the stiffening sleeve 104 from further translating distally through the cannula 212 with the needle 102, and creating external proximal force(s) against the stiffening sleeve 104 to retract the stiffening sleeve 104 into the body 106. While in FIG. 2B, the interaction between the transition 206 and the diaphragm 216 is what is used as the “stop” mechanism, in certain embodiments, the transition 206 may be configured to engage with other features of the cannula assembly 208 to prevent the stiffening sleeve 104 from further translating distally though the cannula 212. For example, the transition 206 can engage with a portion of the hub 210, an indentation 220 of the head 218, and/or a transition 222 between the head 218 and a tube of the cannula 212.

When the user removes the surgical tool 100 from the cannula assembly 208 (e.g., pulls the surgical tool 100 proximally away from the cannula assembly 208), the external force(s) against the stiffening sleeve 104 are removed, and the biasing mechanism acts upon the stiffening sleeve 104 to extend it distally. Referring back now to FIG. 2A, when no proximal forces are acting upon the stiffening sleeve 104, the biasing force(s) of the biasing mechanism cause the stiffening sleeve 104 to extend distally to a fully extended state wherein the stiffening sleeve 104 substantially surrounds the portion of the needle 102 external to the body 106 but for the distal end 114. However, once proximal force(s) that overcome the biasing force(s) are reapplied against the stiffening sleeve 104, the stiffening sleeve 104 begins to retract within the body 106 of the surgical tool 100.

FIG. 3A illustrates a cross-sectional perspective view of a portion of a surgical tool 300 including a magnet-biased stiffening sleeve 304 in an extended state, according to embodiments described herein. The surgical tool 300 and stiffening sleeve 304 are generally similar in function and geometry to the surgical tool 100 and stiffening sleeve 104, respectively, as described with reference to FIGS. 1-2B. The surgical tool 300 includes a body 306, the stiffening sleeve 304, and a needle 302 extending from a distal end 308 of the body 306. A proximal end 324 of the stiffening sleeve 304 extends into a barrel 322 within a central cavity 310 of the body 306. A proximal end 328 of the barrel 322 is at least partially defined by a needle holder 320.

The surgical tool 300 further includes at least a first, distal magnet 312 and a second, proximal magnet 316, which work together to bias the stiffening sleeve 304 distally outward from the body 306. The distal magnet 312 is fixedly attached to the proximal end 324 of the stiffening sleeve 304 and is configured to translate with the stiffening sleeve 304 within the barrel 322. The proximal magnet 316 is fixedly attached to the needle holder 320 at the proximal end 328 of the barrel 322. The proximal and distal magnets 312 and 316 are arranged such that repulsive magnetic force(s) are present between the magnets 312 and 316, biasing them away from one another.

As shown in FIG. 3A, in a resting state, or when external, proximally-directed force(s) on the stiffening sleeve 304 are smaller than the repulsive force(s) of the magnets 312 and 316, the distal magnet 312, and thus, the stiffening sleeve 304 attached thereto, are biased distally toward a distal, fully extended state. In this fully extended state, the distal magnet 312 is pressed against a distal end 314 of the barrel 322, and the stiffening sleeve 304 sheathes substantially all of the portion of the needle 302 external to the body 306 but for a distal end 334 of the needle 302. When the external, proximally directed force(s) on the stiffening sleeve 304 overcome the repulsive forces of the magnets 312 and 316, the stiffening sleeve 304, and thus the distal magnet 312, are translated proximally to a proximal, fully retracted state (shown in FIG. 3C).

To facilitate repelling of the magnets 312 and 316, and thus, distal biasing of the stiffening sleeve 304, the magnets 312 and 316 can be arranged such that like poles of the magnets 312 and 316 are facing each other, or are arranged closest to each other, within the barrel 322, and opposite poles of the magnets 312 and 316 are facing away from each other. For example, in certain embodiments, the distal magnet 312 and the proximal magnet 316 are positioned such that a north pole of each of the distal magnet 312 and the proximal magnet 316 faces, or is arranged closest to, the opposing magnet. For example, the north pole of the distal magnet 312 can be positioned to face towards the proximal magnet 316 and the needle holder 320, while the north pole of the proximal magnet 316 can be positioned to face towards the distal magnet 312 and the distal end 308 of the body 306. When the north poles of the magnets 312 and 316 are facing one another, the magnets 312 and 316 repel each other. Alternatively, in certain embodiments, the south poles of the magnets 312 and 316 can face one another, thereby repelling one another.

In certain embodiments, at least one of the distal magnet 312 and the proximal magnet 316 are welded to the stiffening sleeve 304 and the needle holder 320, respectively. In certain other embodiments, at least one of the distal magnet 312 and the proximal magnet 316 are secured to the stiffening sleeve 304 and the needle holder 320, respectively, with an adhesive or bonding material. In certain embodiments, at least one of the distal magnet 312 and the proximal magnet 316 are secured to the stiffening sleeve 304 and the needle holder 320, respectively, via a mechanical coupling mechanism, such as a snap fit or interference fit.

In certain embodiments, one or both of the magnets 312 and 316 are substantially circular magnets having a central opening through which the needle 302 is disposed, as shown in FIG. 3A. In certain embodiments, the magnets 312 and 316 are formed of multiple smaller magnets secured together to form an annular, ring shaped structure. For example, the magnets 312 and 316 can be formed of many small cylindrical magnets arranged together to create the necessary repulsive forces for biasing the stiffening sleeve 304. The small cylindrical magnets can be secured to one another using an adhesive or bonding agent, or welding, for example, to form a ring-shaped structure suitable to be positioned around an outer circumference of the needle 302.

In certain embodiments, the magnets 312 and 316 are formed of iron, nickel, cobalt, neodymium, or other suitable metallic materials and/or metallic alloys. The magnets 312 and 316 can be coated with gold, rubber, silver, epoxy, polytetrafluoroethylene (PTFE), or other materials to maintain biocompatibility of the magnets for use in the surgical instrument. Though described with particular shapes, dimensions, and/or materials, the magnets 312 and 316 can have any suitable shapes and/or dimensions to fit within the body 306 and/or around the needle 302, and can be formed of any suitable materials, to provide suitable repulsive forces between each other for biasing the stiffening sleeve 304.

As described in reference to FIGS. 2A-2B, the stiffening sleeve 304 is configured to retract in response to external force(s) acting against the stiffening sleeve 304 in the proximal direction overcoming the repelling force(s) of the magnets 312. In certain embodiments, the external force(s) required to overcome the magnet repulsion and retract the stiffening sleeve 304 collectively amount to 1 pound of force or more. In certain embodiments, the external force(s) required to overcome the magnet repulsion collectively amount to 0.5 pounds of force or more. In certain other embodiments, the external force(s) required to overcome the magnet repulsion collectively amount to less than 0.5 pounds of force. The external force(s) can be created by a user inserting the distal end 334 of the needle 302 and the stiffening sleeve 304 into a cannula assembly, as described in reference to FIG. 2.

FIG. 3B illustrates a cross-sectional view of a portion of the surgical tool 300 including the magnet-biased stiffening sleeve 304, according to embodiments described herein. FIG. 3B demonstrates the magnetic fields of magnets 312 and 316 from the north and south poles of each magnet, respectively. The surgical tool 300 in FIG. 3B is shown in a fully extended state wherein the stiffening sleeve 304 is translated to a most distal state and substantially surrounds the portion of the needle 302 external to the body 306, but for the distal end 334.

As described above, in certain embodiments, the north poles (or south poles) of each magnet 312 and 316, respectively, can be facing each other such that the magnetic fields 330 and 332 of the magnets 312 and 316, respectively, repel one another. The north poles of the distal magnet 312 and the proximal magnet 316 are labeled “N” in FIG. 3B. When the magnitude of the external force(s) proximally applied to the stiffening sleeve 304 is less than the magnitude of the repulsive force(s) created by the arrangement of the poles of the magnets 312 and 316, the repulsive force(s) cause the distal magnet 312, and thus the stiffening sleeve 304 attached thereto, to translate distally, away from the proximal magnet 316 and over the needle 302.

FIG. 3C illustrates another cross-sectional view of a portion of the surgical tool 300 including the magnet-biased stiffening sleeve 304, according to embodiments described herein. The surgical tool 300 in FIG. 3C is shown with the stiffening sleeve 304 in a retracted state, wherein the stiffening sleeve 304 only partially surrounding the portion of the needle 302 external to the body 306.

When the opposing external force(s) are greater in magnitude than the repelling force(s) of the magnets 312 and 316, such as when a user inserts the needle 302 into a cannula assembly as described in reference to FIGS. 2A-2B, the stiffening sleeve 304 translates proximally and retracts towards the body 306. This proximal movement of the stiffening sleeve 304 causes at least a portion of the needle 302 to be exposed beyond just the distal end 334. When the stiffening sleeve 304 cannot be further translated in the proximal direction, the stiffening sleeve 304 is in a fully retracted state.

As described above, the magnets 312 and 316 can be configured to create repulsive force(s) therebetween such that 0.5 pounds or 1 pound of opposing force(s) is required to overcome the repulsive forces and retract the stiffening sleeve 304, for example.

The stiffening sleeve 304 can then return to the extended state, as shown in FIGS. 3A and 3B, when the external force(s) applied to the stiffening sleeve 304 are removed. For example, when a surgeon has completed a surgical procedure and begins to remove the needle 302 from the cannula assembly, the stiffening sleeve 304 can then return to the extended state over the needle 302 as a result of the repelling forces between the magnets 312 and 316.

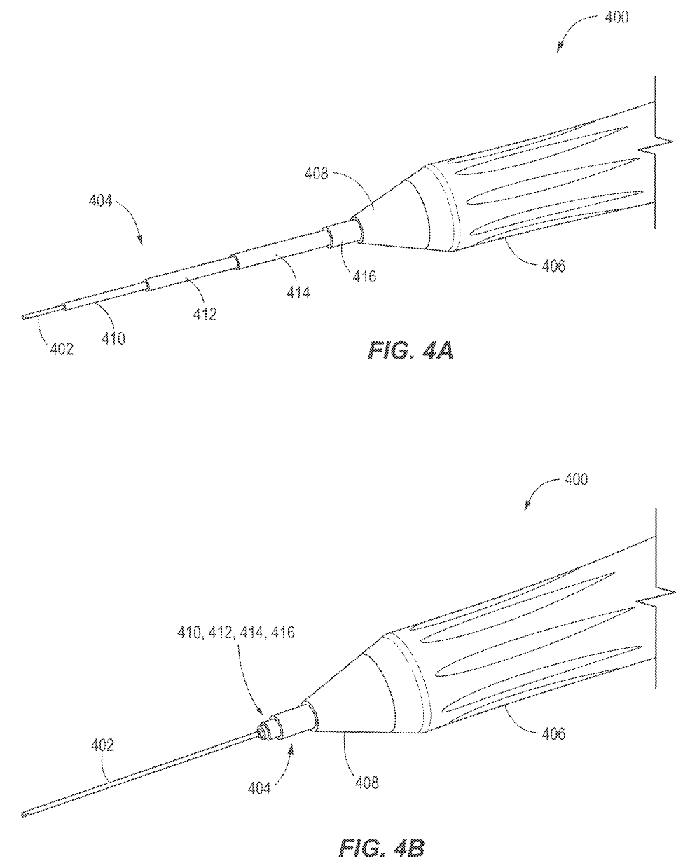

FIG. 4A illustrates an isometric view of a surgical tool 400 having a telescopic stiffening sleeve 404, according to embodiments described herein. The telescopic stiffening sleeve 404, as shown, includes four segments 410, 412, 414, and 416 of increasing diameter, respectively, from a distal end of the telescopic stiffening sleeve 404 to a proximal end of the telescopic stiffening sleeve 404. Each of the four segments 410, 412, 414, and 416 is configured to nest, at least partially, within at least one segment of larger diameter to allow the telescopic stiffening sleeve 404 to retract into a distal end 408 of the surgical tool 400 and expose the needle for a surgical procedure as described herein. Generally, the segments 410, 412, 414, and 416 are tubular in shape.

As shown, the first segment 410 has an outer diameter smaller than an inner diameter of the second segment 412, but larger than an outer diameter of the needle (e.g., a 20 gauge, 23 gauge, 25 gauge, 27 gauge, or smaller, needle). The second segment 412 has an outer diameter smaller than an inner diameter of the third segment 414. The third segment 414 has an outer diameter smaller than an inner diameter of the fourth segment 416, etc. In certain embodiments, an outer surface of the first segment 410 is configured to engage with one or more internal features of a cannula assembly, as described in FIGS. 2A-2B, to retract the stiffening sleeve 404 as the needle 402 is inserted into the cannula assembly. In such embodiments, the outer diameter of the first segment 410 is sized such that the outer surface of the first segment 410 cannot be translated/passed through the cannula of the cannula assembly.

In certain embodiments, one or more of segments 410, 412, 414, and 416 of the telescopic stiffening sleeve 404 can be formed of a metallic material such as surgical grade stainless steel, aluminum, or the like. In certain embodiments, one or more of segments 410, 412, 414, and 416 of the telescopic stiffening sleeve 404 can be formed of a thermoplastic polymeric material, such as high-density polyethylene (HDPE) or the like.

Though the telescopic stiffening sleeve 404 is shown in FIG. 4A as having four segments, the telescopic stiffening sleeve 404 can have any number of segments that allow for the telescopic stiffening sleeve 404 to retract within a body 406 of the surgical tool and extend to sheath at least a majority of a length of the needle extending distally beyond the distal end of the body 406. For example, the telescopic stiffening sleeve 404 can include two, three, five, six, seven, or more segments configured to at least partially nest within each other when the telescopic stiffening sleeve 404 is retracted.

Each of the segments 410, 412, 414, and 416 is slidably coupled to adjoining segment(s) via one or more mechanical features, as described in reference to FIGS. 5A-5B.

Similar to the stiffening sleeves described above, the telescopic stiffening sleeve 404 is configured to retract upon application of external proximal forces against the telescopic stiffening sleeve 404 that overcome a biasing mechanism of the surgical tool configured to control the retraction of the telescopic stiffening sleeve 404. An example biasing mechanism is described in reference to FIGS. 5A-5C.

In certain embodiments, the retraction of the telescopic stiffening sleeve 404 occurs in a step-wise manner. For example, following the application of force(s) to a distal end of the telescopic stiffening sleeve 404 (e.g., against first segment 410), the first segment 410 can be retracted into the second segment 412. Once the first segment 410 is fully retracted into the second segment 412, the first and second segments 410 and 412 can be retracted into the third segment 414. Once the first and second segments 410 and 412 are fully retracted into the third segment 414, the first, second, and third segments 410, 412, and 414 can be retracted into the fourth segment 416.

FIG. 4B illustrates an isometric view of the surgical tool 400 with the telescopic stiffening sleeve 404 in a fully retracted state. As described in reference to FIG. 4A, the four segments 410, 412, 414, and 416 are nested within each other to fully retract the telescopic stiffening sleeve 404 and expose a majority of the length of the needle 402 external to the body 406. In certain embodiments, “fully retracted” can refer to the first, second and third segments 410, 412, and 414 fully nested within the fourth segment 416. In certain embodiments, the fourth segment 416, within which the first, second and third segments 410, 412, and 414 are nested, can further retract into the distal end 408 of the body 406 to expose an entirety of the length of the needle 402 external to the body 406.

FIGS. 5A-5C illustrate cross-sectional views of a portion of the surgical tool 400 having the telescopic stiffening sleeve 404 in an extended state and a retracted state, respectively, according to embodiments described herein. FIGS. 5A-5C demonstrate an example biasing mechanism 418 for facilitating the retraction and extension of the telescopic stiffening sleeve 404 into/from a body 406 of the surgical tool 400.

The biasing mechanism 418 in FIGS. 5A- 5C is illustrated as a compression spring. However, in certain embodiments, other biasing mechanisms, such as the magnets described in reference to FIGS. 3A-4B can alternatively be utilized with the telescopic stiffening sleeve 404. In such an example, a distal magnet can be secured to a proximal end of a first segment 410, and a proximal magnet can be secured to a needle holder 430 within a barrel 432, wherein the magnets are arranged to repel each other.

Where the biasing mechanism 418 includes a compression spring, the biasing mechanism 418 can have a distal end 420 coupled to, or pressed against, the first segment 410 and a proximal end 422 disposed within a body 406 of the surgical tool 400. In certain embodiments, the proximal end 422 of the biasing mechanism 418 is coupled to a needle holder 430 disposed within a barrel 432 of the surgical tool 400. The needle holder 430 is configured to rigidly secure the needle 402 in place. As a result of the arrangement, the biasing mechanism 418 extends along a length of a needle 402 within the second segment 412, the third segment 414, and the fourth segment 416 of the stiffening sleeve 404.

In FIG. 5A, the biasing mechanism 418 is shown in a fully extended state. As such, the biasing mechanism 418 maintains the telescopic stiffening sleeve 404 along almost an entirety of the length of the needle 402 (except for a portion of a distal end of the needle 402).

As referenced above in FIG. 4A and detailed in FIG. 5B, each of the segments 410, 412, 414, and 416 is slidably coupled to adjoining segment(s) via one or more mechanical features to maintain a connection between adjacent segments when the telescopic stiffening sleeve 404 is in an extended position. For example, the first segment 410 includes a flange 434 disposed around an external circumference of the segment 410 at a proximal end of the segment 410. The flange 434 is configured to interact with a lip 436 within an internal circumference at a distal end of the second segment 412. Similarly, the second segment 412 includes a flange 434 around an external circumference of the segment 412 at a proximal end of the segment 412 configured to interact with a lip 436 within the internal circumference at a distal end of the third segment 414. This interaction between flanges 434 and lips 436 of adjoining segments continues along the length of the telescopic stiffening sleeve 404 between adjoining segments.

Though lips 436 and flanges 434 are shown in FIGS. 5A-5B and described herein, other mechanical features such as a groove and a pin, for example, may alternatively be utilized to slidably secure the segments 410, 412, 414, and 416 to one another.

When external proximal force(s) act against one of the segments 410, 412, 414, or 416 and overcomes the distal biasing force of the biasing mechanism 418, that segment retracts into the adjacent and proximal segment while also pulling the adjacent distal segment therewith. This mechanism of the telescoping stiffening sleeve 404 can continue until the segment against which the external force(s) are acting is at least partially retracted within the body 406, as long as the external proximal force(s) are present. Where the external force(s) are applied to the most distal segment 410, the retraction mechanism of the telescoping stiffening sleeve 404 can continue until the telescoping stiffening sleeve is fully retracted, as shown in FIG. 5C. As the telescoping stiffening sleeve 404 retracts, the biasing mechanism 418 compresses, and the segments 410, 412, and/or 414 at least partially nest within the adjacent proximal segments and/or within the body 406.

Upon removal of the external force(s), the biasing mechanism 418 returns to a relaxed state, thereby extending the telescoping stiffening sleeve 404 distally over the needle 402. In certain embodiments, the external force(s) can be created by a user inserting the needle 402 and the telescoping stiffening sleeve 404 of the surgical tool 400 into a cannula assembly during a surgical procedure, as described in reference to FIG. 2. As the user begins to remove the surgical tool 400 from the cannula assembly, the external force(s) as created by the user pressing the surgical tool 400 into the cannula assembly are removed from the telescoping stiffening sleeve 404, and the biasing mechanism 418 is returned to the relaxed state wherein the telescoping stiffening sleeve 404 is extended over the needle 402.

The present disclosure may be embodied in other specific forms without departing from its spirit or essential characteristics. The described embodiments are to be considered in all respects only as illustrative and not restrictive. The scope of the present disclosure is, therefore, indicated by the appended Claims rather than by this Detailed Description. All changes which come within the meaning and range of equivalency of the Claims are to be embraced within their scope.

Reference throughout this specification to features, advantages, or similar language does not imply that all of the features and advantages that may be realized with the present disclosure should be or are in any single embodiment of the disclosure. Rather, language referring to the features and advantages is understood to mean that a specific feature, advantage, or characteristic described in connection with an embodiment is included in at least one embodiment of the present disclosure. Thus, discussions of the features and advantages, and similar language, throughout this specification may, but do not necessarily, refer to the same embodiment.

Furthermore, the described features, advantages, and characteristics of the disclosure may be combined in any suitable manner in one or more embodiments. One skilled in the relevant art will recognize, in light of the description herein, that the disclosure can be practiced without one or more of the specific features or advantages of a particular embodiment. In other instances, additional features and advantages may be recognized in certain embodiments that may not be present in all embodiments of the present disclosure.

Reference throughout this specification to "one embodiment", "an embodiment", or similar language means that a particular feature, structure, or characteristic described in connection with the indicated embodiment is included in at least one embodiment of the present disclosure. Thus, the phrases "in one embodiment", "in an embodiment", and similar language throughout this specification may, but do not necessarily, all refer to the same embodiment.

The detailed description and the drawings are supportive and descriptive of the disclosure, but the scope of the disclosure is defined solely by the claims. While some of the best modes and other embodiments for carrying out the claimed disclosure have been described in detail, various alternative designs and embodiments exist for practicing the disclosure defined in the appended claims.

Furthermore, the embodiments shown in the drawings or the characteristics of various embodiments mentioned in the present description are not necessarily to be understood as embodiments independent of each other. Rather, it is possible that each of the characteristics described in one of the examples of an embodiment can be combined with one or a plurality of other desired characteristics from other embodiments, resulting in other embodiments not described in words or by reference to the drawings. Accordingly, such other embodiments may fall within the scope of the appended claims.

What is claimed is:

1. A surgical tool, the surgical tool comprising:
a surgical tool body comprising a proximal end and a distal end;
a needle extending from the distal end of the surgical tool body configured to access a surgical area within a body of a patient;
a stiffening sleeve extending from the distal end of the surgical tool body and surrounding at least a portion of the needle for stabilizing the needle during a surgical procedure;
a first magnet coupled to a proximal end of the stiffening sleeve; and
a second magnet disposed within the surgical tool body, wherein:
the first magnet and the second magnet are arranged such that a repulsive force is formed between the first magnet and the second magnet,
when external proximal forces on the stiffening sleeve are less than the repulsive force, the first magnet and the stiffening sleeve are in a fully extended position, and
when external proximal forces on the stiffening sleeve are greater than the repulsive force, the first magnet and the stiffening sleeve are retracted proximally towards the surgical tool body.

2. The surgical tool of claim 1, wherein the first magnet is configured to remain within a central cavity of the surgical tool body when the stiffening sleeve is in a retracted and an extended position.

3. The surgical tool of claim 1, wherein the surgical tool comprises a vitrectomy probe, an illumination probe, forceps, or scissors.

4. The surgical tool of claim 1, wherein the first magnet and the second magnet are positioned such that like poles of the first magnet and the second magnet face each other.

5. The surgical tool of claim 1, wherein each of the first magnet and the second magnet is a substantially circular magnet with a central opening, wherein the needle is positioned within the central opening of the first magnet and the central opening of the second magnet.

6. The surgical tool of claim 1, wherein each of the first magnet and the second magnet comprises a plurality of cylindrical magnets forming a ring-shaped structure, wherein the needle is positioned within central openings of the ring-shaped structure of the first magnet and the ring-shaped structure of the second magnet.

7. The surgical tool of claim 1, wherein the external proximal forces required to overcome the repulsive force between the first magnet and the second magnet are between 0.5 pounds to 1 pound of force.

8. A surgical tool, the surgical tool comprising:
a surgical tool body comprising a proximal end and a distal end;
a needle extending from the distal end of the surgical tool body configured to access a surgical area within a body of a patient; and
a telescopic stiffening sleeve extending from the distal end of the surgical tool body and surrounding at least a portion of the needle for stabilizing the needle during a surgical procedure, the telescopic stiffening sleeve comprising at least a first segment and a second segment; and
a biasing mechanism disposed within the surgical tool body and extending within the telescopic stiffening sleeve, wherein:
the biasing mechanism is configured to provide a distal biasing force against the telescopic stiffening sleeve,
when external proximal forces on the telescopic stiffening sleeve are less than the distal biasing force, the telescopic stiffening sleeve is fully extended,
when external proximal forces on the telescopic stiffening sleeve are greater than the distal biasing force, the telescopic stiffening sleeve is retracted proximally towards the surgical tool body, and
the first segment is configured to at least partially nest within the second segment as the telescopic stiffening sleeve is retracted proximally.

9. The surgical tool of claim 8, wherein the surgical tool further comprises a third segment within which the first segment and the second segment are configured to at least partially nest as the telescopic stiffening sleeve is retracted proximally.

10. The surgical tool of claim 9, wherein the surgical tool further comprises a fourth segment within which the first segment, the second segment, and the third segment are configured to at least partially nest as the telescopic stiffening sleeve is retracted proximally.

11. The surgical tool of claim 8, wherein the biasing mechanism comprises a compression spring.

12. The surgical tool of claim 11, wherein a proximal end of the biasing mechanism is secured within the surgical tool body and a distal end of the biasing mechanism is secured to a proximal end of the first segment of the telescopic stiffening sleeve.

13. The surgical tool of claim 12, wherein the biasing mechanism extends along a length of the needle within the telescopic stiffening sleeve.

14. The surgical tool of claim 8, wherein the surgical tool comprises a vitrectomy probe, an illumination probe, forceps, or scissors.

15. The surgical tool of claim 8, wherein:
the first segment includes a flange disposed around an external circumference of the first segment at a proximal end of the first segment,
the second segment includes a lip within an internal circumference at a distal end of the second segment, and
the flange of the first segment is configured to interact with the lip of the second segment to maintain a connection between the first segment and the second segment when the telescopic stiffening sleeve is fully extended.
